(19) United States
(12) Patent Application Publication
(10) Pub. No.: US 2013/0331735 A1
(43) Pub. Date: Dec. 12, 2013

(54) INTERVENTIONAL GUIDEWIRE

(71) Applicant: Henry W. Lupton, Oranmore (IE)

(72) Inventor: Henry W. Lupton, Oranmore (IE)

(21) Appl. No.: 13/992,891

(22) PCT Filed: Dec. 14, 2011
(86) PCT No.: PCT/US11/64893

§ 371 Date: Aug. 27, 2013

Related U.S. Application Data
(60) Provisional application No. 61/422,969, filed on Dec. 14, 2010.

(51) Int. Cl. A61B 17/34; A61B 5/00

(52) U.S. Cl. CPC A61B 17/3415; A61B 5/6851
USPC 600/585; 606/159

(57) ABSTRACT

The present invention is an interventional guidewire particularly adapted for use in percutaneous transluminal angioplasty (PTA) procedures. A guidewire of this invention enhances peripheral vascular access for either diagnostic or therapeutic vascular or arterial procedures by having opposing ends of different stiffnesses. Retrograde dorsalis pedis artery access to upper body vascular occlusions is a particularly preferred application of this invention.

The present invention is an interventional guidewire particularly adapted for use in percutaneous transluminal angioplasty (PTA) procedures. A guidewire of this invention enhances peripheral vascular access for either diagnostic or therapeutic vascular or arterial procedures. A guidewire of this invention is particularly useful for procedures in which the site or sites of medical interest can be accessed by the dorsalis pedis artery.

Access to distal limb vascular structure to perform therapeutic or diagnostic procedures at a site of medical interest can be difficult. Such vascular structures often include diffuse calcification and, on occasion, chronic total occlusions. Conventional femoral access procedures are accomplished using antegrade (i.e., in the direction of blood flow) and retrograde (i.e., in the opposite direction as blood flow) using a range of guidewires of varying diameter. Guide catheters are then threaded or slid over the operant guidewire to the site of medical interest, e.g., a balloon catheter to perform vessel angioplasty. For various reasons, rather than femoral access, lower limb vascular access is being obtained more commonly by means of the dorsalis pedis artery (see FIG. 1, below).

Vascular access by means of the dorsalis pedis artery has the significant drawback that the artery is of a smaller diameter than the femoral arteries and can only accommodate a relatively smaller diameter needle and thus a smaller diameter interventional guidewire.

To overcome this problem, one approach that has been used is that the tip of the wire is exteriorized at the femoral site once the occluded segment is passed. The wire is then replaced with a second wire coming from the region with its tip directed to the distal limb.

### BRIEF SUMMARY OF THE INVENTION

Briefly, in one aspect, the present invention overcomes the need to use multiple guidewires in a single procedure to obtain lower limb vascular access. A guidewire of this invention is generally structurally symmetric end-to-end meaning essentially that its two ends are similar in structure and appearance (though not necessarily identical) with the exception that one end is relatively softer than the other. Put another way, one end of the otherwise structurally similar guidewire is stiffer than the other. In basic terms, the present guidewire is a double-ended guidewire having one relatively stiffer end and one relatively softer end.

In one embodiment of this invention, a double-ended guidewire having relatively softer and relatively stiffer ends is a single guidewire structure.

In a second embodiment of the invention, the double-ended guidewire with relatively stiffer and softer ends is created from two different guidewire segments coupled, e.g., by means of a coupling tube. In this embodiment, the guidewires are connected at their reduced diameter, usually threaded, coupler ends using, for example a turnbuckle or other coupling structure. This embodiment of the invention provides some additional flexibility in use of the relatively softer and relatively stiffer guidewire segments for both catheter selection and placement.

In a further aspect, the present invention is a method of crossing an occluded vessel for therapeutic or diagnostic purposes wherein the occlusion is sufficiently rigid such that a therapeutic guidewire cannot cross the occlusion without either or both of vessel injury or guidewire damage, comprising the steps of:
deploying a first guidewire into the occluded vessel upstream from the occlusion wherein the distal end of the guidewire is positioned immediately adjacent the upstream portion or segment the occlusion, the guidewire having itself, or providing to a separate device means to connect to a guidewire emerging through the occlusion from the downstream portion or segment of the occlusion;
deploying a second guidewire downstream from the occlusion and directing the distal end of the downstream guidewire to and through the occlusion;
crossing the occlusion from the downstream direction with the second guidewire;
connecting the distal end of the downstream guidewire to the first guidewire or separate device;
pulling the distal end of the downstream guidewire through the occlusion to where the distal end of the downstream guidewire can be accessed to pass a catheter thereover adjacent to the site of the occlusion.

Guidewires of this invention are referred to as “double-ended”. That terminology is intended to mean or refer to a guidewire, as that term is understood in the vascular intervention art, in which or on which both the distal end and proximal end of the guidewire have substantially the same structural elements and, usually, in many but not all cases appear similarly.

In use, the relatively stiffer end of the guidewires is inserted into the dorsalis pedis artery and steered across the foot and up the lower limb into the femoral artery. The softer end of the guidewire is positioned in the vessel at the site of medical interest. The relatively stiffer guidewire segment now becomes the proximal end of the guidewire (from the patient's perspective) and provides the structure for the instruction of guide catheter into the vessel. The relatively softer guidewire end remains adjacent the site of medical interest and provides, for example, a softer guidewire support for dilatation using e.g., a balloon catheter.

In a further aspect, femoral artery access is obtained using, for example, an introducer sheath and, perhaps, a catheter such as a guide catheter. The distal end of the operant device is directed through the arterial or vascular system to the site of the blockage. In at least some instances the blockage site is a chronic total occlusion that cannot be easily “crossed” without vascular or device damage. Experience has shown that the downstream (in the sense of blood flow) end or segment of the blockage is generally less calcified and is therefor easier to cross. A second vascular access then is obtained via the pedal artery and a smaller diameter (i.e., 0.018 in or less) interventional wire of this invention is used to approach the blockage from the softer downstream side. Presuming the blockage is successfully crossed from the downstream side using the smaller diameter wire, the distal end of that smaller diameter wire then is connected to or coupled to the initially-placed guidewire (such as being snared by means of a loop on a guide catheter) and is drawn through the blockage to the point where the stiffer end exits from the femoral site. The total or at least difficult-to-cross occlusion having now been successfully crossed can then be dilated and or stented by means of a catheter passed over the secondly placed guidewire with the softer end of the guidewire being located adjacent the blockage as is preferred for such procedures.

### BRIEF DESCRIPTION OF THE DRAWINGS

FIG. 1, which was published in Patent Application Publication US2009/0018566 to Escudero et al. is a view of a limb, including the lower limb, showing the detailed vascular and arterial structures to which the present invention can be applied;

FIG. 2 illustrates a test mechanism used to measure guidewire stiffness according to this invention.

FIG. 3 shows stiffness measurement results obtained using the mechanism of FIG. 2.

FIG. 4 is a detailed illustration of one embodiment of this invention.

FIG. 5 is a schematic illustration of a first double-ended embodiment of the present invention which comprises a single guidewire structure;

FIG. 6 is a second schematic illustration of an embodiment of the invention showing two guidewire segments coupled by means of reduced diameter coupler segment.

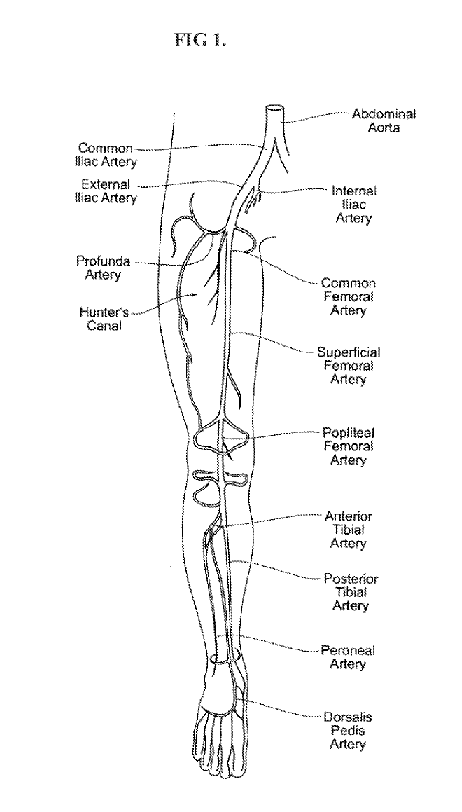

It is noted that the present invention contemplates the utilization of smaller diameter, interventional guidewires which are double-ended and which have first and second ends with differing stiffness. Techniques to control guidewire stiffness will be well known to those skilled in this art in view of the present disclosure. For example, a series of tapers may be created in one end or the other of a guidewire of this invention (to make it softer), e.g., by centerless grinding. Axial slices or cuts partially through the guidewire core wire are another technique used to increase guidewire softness. Deployment of distal coils, polymer sleeves, and partial heat treatment are other approaches that can be used to create differential guidewire stiffness according to this invention.

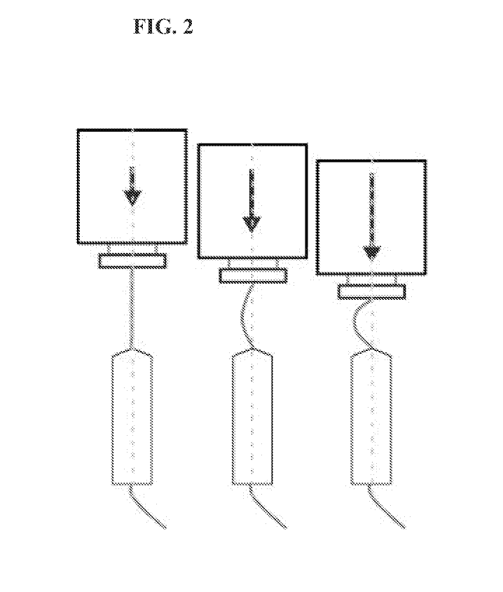

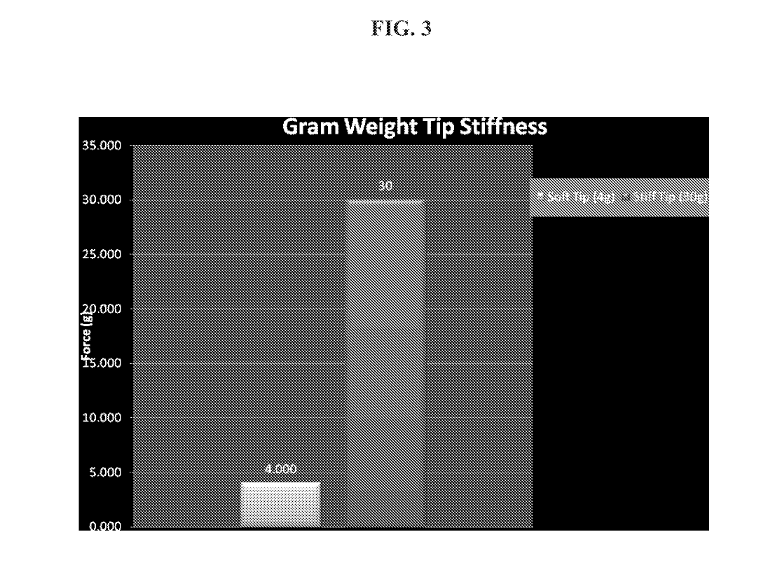

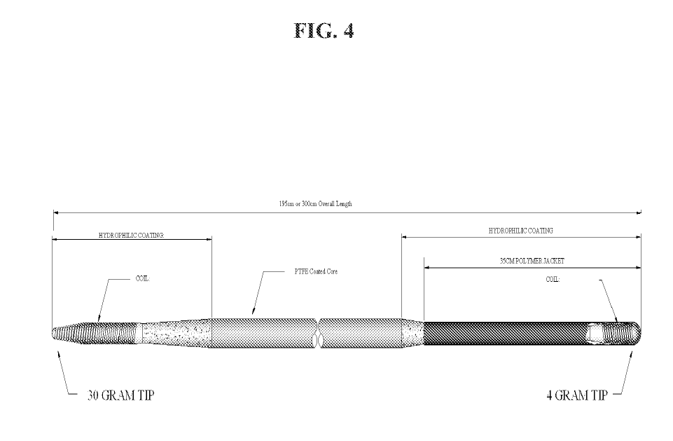

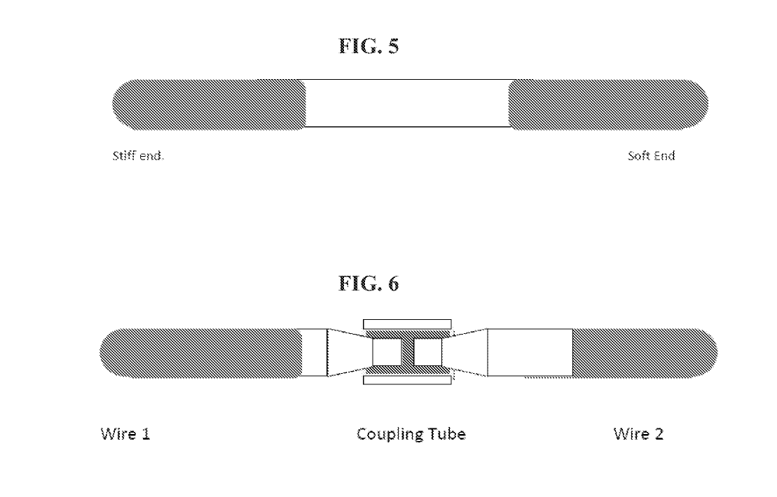

Description of Guidewire Stiffness Measurement: The objective of this test is to determine the gram weight stiffness of the distal tip of a guidewire. In this test the guidewire is clamped at the crosshead and deflected against a balance pan a distance of 0.6 mm. The gram weight is recorded. The test is repeated 3 times and the average is calculated. A depiction of this measurement set up is shown in FIG. 2.

What is claimed is as follows:

1. A double-ended guidewire having a first relatively stiff end and a second relatively softer end.

2. A guidewire according to claim 1 wherein the first end has a stiffness of 4 g.

3. A guidewire according to claim 1 wherein the second end has a stiffness of 30 g.

4. A guidewire according to claim 1 wherein the difference between the stiffness of the first and second ends of the guidewire is at least 2 g.

5. A guidewire according to claim 1 wherein the difference between the stiffness of the first and second ends of the guidewire is at least 20 g.

6. A guidewire according to claim 1 wherein the difference between the stiffness of the first and second ends of the guidewire is at least 25 g.

7. An interventional, double-ended guidewire having opposing ends with different stiffnesses wherein the difference between the stiffnesses of the guidewire opposite ends is at least 25 g.

8. A guidewire according to claim 7 wherein the difference between the stiffnesses of the guidewire opposite ends is at least 20 g.

9. A guidewire according to claim 7 wherein the difference between the stiffnesses of the guidewire opposite ends is at least 25 g.

10. A method of crossing an occluded vessel for therapeutic or diagnostic purposes wherein the occlusion is sufficiently rigid such that a therapeutic guidewire cannot cross the occlusion without either or both of vessel injury or guidewire damage, comprising the steps of:
deploying a first guidewire into the occluded vessel upstream from the occlusion wherein the distal end of the guidewire is positioned immediately adjacent the upstream portion or segment the occlusion, the guidewire having itself, or providing to a separate device means to connect to a guidewire emerging through the occlusion from the downstream portion or segment of the occlusion;
deploying a second guidewire downstream from the occlusion and directing the distal end of the downstream guidewire to and through the occlusion;
crossing the occlusion from the downstream direction with the second guidewire;
connecting the distal end of the downstream guidewire to the first guidewire or separate device;
pulling the distal end of the downstream guidewire through the occlusion to where the distal end of the downstream guidewire can be accessed to pass a catheter thereover adjacent to the site of the occlusion.

11. A method according to claim 10 wherein the first guidewire has a diameter of about 0.035 inches.

12. A method according to claim 10 wherein the second guidewire is a double-ended guidewire having one end with a relatively greater stiffness and a second end with a relatively lesser stiffness.

13. A method according to claim 10 wherein the second guidewire has an overall diameter of less than about 0.018 inches.

14. A method according to claim 10 wherein the first guidewire is deployed into the femoral artery and the second guidewire is deployed into the dorsalis pedis artery.

15. A method according to claim 10 which further includes the steps of passing a therapeutic catheter over the distal end of the downstream guidewire to the site of the occlusion and performing a therapeutic procedure.
